(19) United States
(12) Patent Application Publication
(10) Pub. No.: US 2005/0130309 A1
(43) Pub. Date: Jun. 16, 2005

(54) Detection of silanol groups on a surface

(76) Inventor: Douglas T. Gjerde, Saratoga, CA (US)

Correspondence Address: PhyNexus, Inc.;Attn: IP Dept., Suite A, 3670 Charter Park Dr., San Jose, CA, 95136, US

(21) Appl. No.: 10/733,685

(22) Filed: Dec. 10, 2003

(51) Int. Cl. G01N 33/00

(52) U.S. Cl. 436/34

(57) ABSTRACT

Methods and reagents for the detection and/or quantification of silanol groups on a silica surface are provided. In certain embodiments, the subject methods are used to evaluate the channel surface of a silica capillary tubing. This evaluation can facilitate methods for derivatizing and/or chemically modifying such surfaces.

### FIELD OF THE INVENTION

This invention relates generally to the detection and/or quantification of silanol groups on a silica surface.

### BACKGROUND OF THE INVENTION

A variety of useful devices and instruments include a silica (i.e, silicon dioxide) surface. Examples include silica capillary tubes, silica chips, and silica chromatographic media. Fused silica capillary tubes in particular find utility in a variety of contexts, including capillary electrophoresis, chromatography, solid-phase extraction and other modes of chemical analysis.

A silica surface typically contains a number of free silanol groups (—SiOH) capable of reacting with a fluid medium in contact with the surface, i.e., reactable silanol groups. The concentration of these groups can dramatically affect the functional characteristics of the surface. For example, reactable silanol groups can provide chemically reactive sites useful for the derivitization of the surface, e.g, by the covalent or non-covalent attachment of molecules or other substrates to the surface through the silanol group. As another example, the presence of reactable silanol groups can influence electro-osmotic flow in capillary electrophoresis.

Thus, for a variety of reasons methods and reagents for the detection and/or quantification of reactable silanol groups on a silica surface are of interest. The present invention provides for this need.

### SUMMARY OF THE INVENTION

The subject invention provides a method for measuring the concentration of reactable silanol groups on a silica surface comprising the following steps:
a) contacting a silica surface with a silanol titration solution, wherein said titration solution comprises a detectable and quantifiable titration reagent that binds with substantially all reactable silanol groups on the silica surface;
b) allowing said titration reagent to bind substantially all of the silanol groups on the silica surface;
c) removing the silanol titration solution from the silica surface, along with substantially all of the titration reagent that has not bound to a reactable silanol group, under conditions where titration reagent that has bound to a silanol group remains bound;
d) detecting and quantifying the bound detection reagent, after optionally eluting the bound detection reagent from the silica surface; and
e) determining the concentration of reactable silanol groups on said silica surface from the quantity of bound titration reagent.

In one embodiment, the titration reagent binds substantially all reactable silanol groups on the silica surface in a manner that is substantially stoichiometric, and the known binding stoichiometry between said titration reagent and reactable silanol groups is used to calculate the concentration of reactable silanol groups on said silica surface. It is sometimes desirable to use a titration reagent for which the binding stoichiometry between the titration reagent and the reactable silano groups is known, e.g., a binding stoichiometry of 1:1.

In an embodiment, the silica surface comprises fused silica.

In an embodiment, the silica surface comprises the channel surface of a capillary.

In an embodiment, the titration reagent binds to the reactable silanol groups through an ionic interaction.

In an embodiment, the titration reagent is small enough such that the binding of a first titration reagent to a reactable silanol group on said silica surface does not substantially block any other reactable silanol groups on said silica surface from reaction with a second titration reagent.

In an embodiment, the titration reagent has a MW of less than 500 Da.

In an embodiment, the titration reagent comprises a quaternary alkyl ammonium group, e.g, a benzyltrimethylammonium salt such as benzyltrimethylammonium chloride.

In an embodiment, the titration reagent comprises a chromophore which is used to detect and quantify the bound titration reagent, optionally after elution of the reagent from the silica surface. A preferred chromophore is one that absorbs UV radiation.

In an embodiment, the binding reaction between said titration reagent and a reactable silanol group is reversible, and wherein bound titration reagent is eluted from the silica surface between steps (c) and (d).

In an embodiment, the silica surface is washed to substantially remove the silanol titration solution from the silica surface prior to detection and quantification of bound titration reagent.

In a preferred embodiment, the titration reagent comprises a quaternary alkyl ammonium group and a UV chromophore, wherein the silica surface is washed to substantially remove the silanol titration solution from the silica surface prior to detection and quantification of bound titration reagent, and wherein the titration reagent is detected by absorbance of UV light. A preferred titration reagent is benzyltrimethylammonium.

### DESCRIPTION OF SPECIFIC EMBODIMENTS OF THE INVENTION

Methods and reagents for the detection and/or quantification of reactable silanol groups on a silica surface are provided.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a molecule” includes a plurality of such molecules and reference to “the detection method” includes reference to one or more detection methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In accordance with the present invention there may be employed conventional chemistry, biological and analytical techniques within the skill of the art. Such techniques are explained fully in the literature. See, e.g. Chromatography, 5^{th} edition, PART A: FUNDAMENTALS AND TECHNIQUES, editor: E. Heftmann, Elsevier Science Publishing Company, New York, pp A25 (1992); ADVANCED CHROMATOGRAPHIC AND ELECTROMIGRATION METHODS IN BIOSCIENCES, editor: Z. Deyl, Elsevier Science BV, Amsterdam, The Netherlands, pp 528 (1998); CHROMATOGRAPHY TODAY, Colin F. Poole and Salwa K. Poole, and Elsevier Science Publishing Company, New York, pp 394 (1991).

As summarized above, the subject invention provides reagents for the detection and/or quantification of silanol groups on a surface, as well as methods for their use and kits that include the subject compounds.

The subject invention provides a method for measuring the concentration of reactable silanol groups on a silica surface comprising the steps of:
a) contacting a silica surface with a silanol titration solution, wherein said titration solution comprises a detectable and quantifiable titration reagent that binds with substantially all reactable silanol groups on the silica surface;
b) allowing said titration reagent to bind substantially all of the silanol groups on the silica surface;
c) removing the silanol titration solution from the silica surface, along with substantially all of the titration reagent that has not bound to a reactable silanol group, under conditions where titration reagent that has bound to a silanol group remains bound;
d) detecting and quantifying the bound detection reagent, after optionally eluting the bound detection reagent from the silica surface; and
e) determining the concentration of reactable silanol groups on said silica surface from the quantity of bound titration reagent.

In certain embodiment of the present invention the silica surface is the channel wall of a capillary tubing, e.g., a fused silica capillary tubing. Such tubing finds utility in a variety of applications, such as analytical chemistry (e.g., gas chromatography, liquid chromatography, capillary electrophoresis and solid-phase extraction), fluid delivery, drug delivery systems, flow cells, flow restrictors, micro-pipettes, fluid filled or hollow wave guides, micro insulators, coupling ferrules, and micro-optical elements. See, for example, U.S. patent application Ser. No. 10/434,713 and references cited therein. A variety of reviews relevant to the use of silica capillary tubing are available, see for example High Resolution Gas Chromatography, 2^{nd} Ed, 1981, R. R. Freeman and High Performance Capillary Electrophoresis—An Introduction, Dr. David N. Heiger, Hewlett Packard GmbH, Waldbronn, Germany, 1992.

As used herein the term “fused silica” refers to silicon dioxide (SiO₂) in its amorphous (glassy) state, which is a species of the broader genera of compositions commonly referred to as “glass.” Preferred capillaries of the instant invention are produced using high quality synthetic glass of nearly pure SiO₂ The term “synthetic fused silica” refers to amorphous silicon dioxide that has been produced through chemical deposition rather than refinement of natural ore. This synthetic material is of much higher purity and quality as compare to fused quartz made from natural minerals. Examples of fused silica capillaries relevant to this invention include those produced by Polymicro Technologies, LLC of Phoenix, Ariz. and SGE Inc. of Ringwood, Australia.

In a variety of applications, the free silanol groups on the channel surface of a silica capillary tubing serve as attachment points for the covalent or non-covalent attachment of chemical entities. See, for example, U.S. patent application Ser. No. 10/434,713, which describes the chemical modification of silica capillaries to introduce or modify extraction surfaces on the channel surface of the capillary. The silanol groups on the silica surface serve as useful attachment points for the extraction chemistries.

Using the methods and reagents described herein, we have shown that the amount of free silanol groups in comparable capillaries can vary substantially, depending upon factors such as the source of the capillary, the particular lot, and on how the capillary has been treated (see the Examples appended hereto). For example, etching of a capillary (e.g., by treatment with base) can result in marked increase in number of the silanol groups. The number of reactable silanol groups, in turn, can be a factor in determining the capacity of the surface for chemical modification. Where the modification relies on covalent derivatization of silanol groups, the degree of modification that can be achieved might be limited by the amount of reactable silanol groups that are available for reaction.

Thus, in the context of silica capillary tubing, there are a number of potentially important applications of the subject invention. By assaying for silanol groups, one can compare different lots of capillaries, from either the same or different vendors, to determine if there are differences in silanol concentration, and in particular which lots or vendors provide the desired amount of silanol groups. Using this information, one might want to choose one vendor over another. Alternatively, by being able to monitor silanol concentration it might be possible to modify production or handling methods to achieve better levels of reactable silanol groups.

In another embodiment, the method can be used to determine the effectiveness of a procedure used to modify a silica surface, e.g., etching of a fused silica capillary with base. As shown in the Examples, etching can dramatically increase the number of reactable silanol groups.

In another embodiment, the method can be used to evaluate a chemical modification of a silica surface, e.g., by covalent attachment of an extraction chemistry to the surface through free silanol groups. For example, the number of free silanol groups in a capillary can be determined before and after chemical modification, and the difference used as a measure of extent of reaction. Alternatively, the number of free silanol groups can be determined prior to modification, and then the extent of modification determined by measuring the presence of a functional group in the modifying reagent. For example, if the modifying group includes a thiol moiety, extent of modification can be determined by assaying for thiol content, e.g., by reaction with DTNB and spectrophometric quantification of the colored product. In this way, one can determine any correlation between number of reactable silanol groups and extent of modification.

As used herein, the term “reactable silanol groups” refers to silanol groups that are on the silica surface and positioned in a manner such that they are available for chemical reaction. Silanol groups that are not reactable are not detected by this method, and are in most instances not relevant since they will not participate in reactions with modifying groups or otherwise effect the chemistry at the silica surface.

There are a number of reasons why it is valuable to be able to determine the concentration of reactable silanol sites on the channel wall of silica capillary tubing. For example, in many instances it is desirable to be able to attach chemical moieties to the wall of a capillary channel through free silanol groups. These moieties can take a variety of forms, e.g., organic groups, biomolecules, affinity groups, etc. These groups can be useful for performing chromatography and solid-phase extractions, as described in more detail in U.S. patent application Ser. No. 10/434,713. By using the subject invention, one can determine the number of free silanol groups prior to derivatization. This will allow one to quantify how many sites are available for attachment and hence predict the amount of derivitization groups that can be attached. This prediction can be compared to the actual number of groups attached to calculate the efficiency of the attachment reaction.

In one embodiment, the subject invention can be used to monitor the concentration and conditions needed for attachment of a silane molecule to a silanol group. In this embodiment, the surface concentration of the starting silanol groups is measured, the silanization reaction is performed under controlled conditions, and then the concentration of the product silane molecule is measured.

If desirable, the number of reactable silanol sites on a surface can be modified to achieve an appropriate concentration for the intended use of the channel. For example, the introduction of more reactable silanol groups on a surface will allow for the attachment of more derivatization groups. Additional silanol group can be introduced by etching the silica surface, e.g., by treatment with base. Thus in one embodiment the subject invention is used to determine the concentration of silanol groups prior to derivitization. If the concentration is lower than desired, etching can often be used to improve the number. The increase in silanol groups as a result of this treatment can be determined by again using the silanol quantification methods of the subject invention. After attachment of the chemical moiety (or during the course of the reaction) the extent of the reaction can be monitored by measuring silanol concentration using the subject invention.

Another advantage of the invention is that it permits one to assess the quality and consistency of silica capillary tubing. Ideally, capillary tubing provided by a vendor will have a sufficient number of silanol sites, and this concentration should be consistent both throughout the length of the tubing and also from one lot of tubing to the next. Without the ability to monitor silanol concentration, one using the tubing might be unaware of inconsistencies in the tubing that could result in inconsistent functioning of the tubing. For example, variability in the number of reactable silanol groups from one lot of tubing to the next could result in variable derivitization of the tubing and hence variable function. Thus, in one embodiment the invention provides a means for controlling and assuring the quality of capillary tubing.

In a related embodiment, the method can be used to improve the manufacturing process. That is, monitoring silanol concentration facilitates the development of manufacturing processes that result in the production of fused silica tubing that has consistent and sufficient reactable silanol groups. The effect of the various methods of tube drawing and etching on silanol group production can be measured. This will allow for the avoidance of unnecessary base etching to achieve adequate silanol sites, which is known to weaken the tube wall and cause the tubing to break easier.

The titration reagent can be any molecule that is capable of quantitatively binding to reactable silanol groups on a silica surface. Preferably, it is a molecule that is small enough that it's binding to a silanol group will not shield adjacent silanol groups on the surface from interacting with the titration reagent. That is, the titration reagent should be a small enough such that its binding to a reactable silanol group does not substantially block any other reactable silanol group on the surface from reaction with another molecule of the titration reagent. Preferably, the titration reagent has a molecular weight (MW) of less than 1000 Da, more preferably less than 500, and still more preferably less than 250. Of course, depending upon the steric properties of the molecule, e.g., it's 3-dimensional structure, in some cases larger molecules will function effectively.

In addition, it is preferable that the titration reagent include a group that enables detection and quantification of the titration reagent. The benzyltrimethylammonium ion, e.g., benzyltrimethylammonium chloride (BTA) is a relatively small molecule that contains an aromatic ring for UV detection, and hence is a good choice. The size of the ammonium moiety does not interfere with the ion exchange process. Furthermore, the molecule reacts specifically with the silanol group under appropriate conditions, e.g., in the presence of methanol. Furthermore, the molecule can be eluted under appropriate conditions, e.g., with 0.1 M NaOH, or 0.1 M HCl and phosphate-buffered saline (PBS) buffer. The molecule absorbs strongly at 254 nm.

In general, a wide variety of titration reagents can be used. Some preferred examples include cations, which bind to reactable silanol groups through an ionic interaction. Examples are amines that are positively charged under the conditions used in the titration, e.g., in the solvent or solvents used. The amine can be primary, secondary, tertiary or quaternary amines, including alkyl quaternary amines such as BTA. Other preferred titration reagents would include certain metal ions, such as Ni²⁺, Fe³⁺, Ag⁺, Na⁺, and others that would be apparent to one of skill in the art. Virtually any metal that does not hydrolyze under the solvent conditions being used for the test can be employed. If a metal or metal derivative reagent is used, a monovalent reagent reacting with one silanol group is preferred.

When an amine or other cationic titration reagent is used, the molecule should include at least one R group that is detectable. For example, this group can be an aromatic ring, e.g., a benzene derivative such as aniline or pyridine, or a detectable functional group having conjugated double bonds capable of fluorescence, e.g., anthracene, phenanthrene, naphthalene, etc.

The silanol titration solution comprises the titration reagent in a solvent in which the reagent is soluble, and in which the reagent binds quantitatively to reactable silanol groups. Typically the binding should be electrostatic as opposed to adsorptive, and the solvent should be chosen accordingly. Thus, where the titration reagent is charged and the interaction electrostatic, the polarity of the solvent should be such that it promotes electrostatic interaction, e.g., of low to medium polarity. Electrostatic interactions are enhanced by reduced polarity of the solvent, and reduced when the solvent is highly polar and/or contains salt or other charged constituent. For example, this can be achieved with BTA by using a low MW alcohol such as methanol as the solvent. Other solvents of similar polarity could also be used provided the titration reagent is sufficiently soluble in the solvent, e.g. another alcohol. In some embodiments an aqueous solvent is sued with an organic solvent wash. Regardless, the solvent must support the ionic state of the silanol and the titration reagent.

Where the silica surface is the surface of a fused silica capillary channel, titration reagent can be conveniently contacted with the surface by flowing it through the capillary, e.g., by pumping it through at a rate that allows for quantitative binding. After being allowed to react, the titration solution is removed from the silica surface, along with any unreacted titration reagent. This can be accomplished, for example, by pumping or otherwise displacing the solution from a capillary. It is generally desirable to wash the surface to more effectively remove unreacted titration reagent, e.g., by flowing a liquid through the capillary. It is convenient to use the medium in which the titration reagent is dissolved, e.g., methanol in the case of a titration solution comprising a titration reagent dissolved in methanol. After washing the surface, any remaining liquid can optionally be removed, e.g., by blowing gas through a capillary to substantially remove any remaining liquid. Care should be taken such that any titration reagent that is specifically bound to a silanol group is not removed during any washing or liquid removal steps.

In some embodiments, a titration reagent binds substantially all reactable silanol groups on the silica surface in a manner that is substantially stoichiometric, such that the known binding stoichiometry between the titration reagent and reactable silanol groups can be used to calculate the concentration of reactable silanol groups on the silica surface. Preferably, the binding stoichiometry between the titration reagent and the reactable silano groups is known, e.g., a 1:1 binding stoichiometry. In order to achieve a 1:1 binding stoichiometry, the reagent radius should not be larger than the midpoint spacing between the silanol groups.

In some preferred embodiments, the reaction between the titration reagent and the silanol group is reversible, and the bound titration reagent is eluted from the silica surface prior to its detection and quantification. The elution conditions should be such that displacement and collection of the reagent is substantially quantitative. In the case where the silica surface is the channel of a silica capillary tubing, elution can be achieved by flowing a desorption solution through the capillary, preferably after washing and drying the channel to substantially remove any unbound titration reagent. Elution of the bound titration reagent can facilitate its detection and quantification, e.g., by detecting the absorbance or fluorescence of the reagent.

Detection can be achieved by any of a number of techniques known in the art. As exemplified in the examples, one can use a titration reagent with a detectable chromophore, e.g., a group that absorbs UV radiation. Other modes of detection could also be used, such as visible, atomic absorption, atomic emission, flame emission detection, fluorescence or mass spectrometry. GC detection can be used if the material is inected and detected by a GC detector wuch as FID. The reagent may detected directly or a detection agent such as a color forming reagent may be added to make the displacement reagent detectable. Fluorescence molecules (molecules that have a planar structure such as naphthalene) to which an alkyl ammonium functional group has been attached may also be used.

The amount of bound titration reagent is determined by detecting the reagent in a manner that allows for its quantification. This can be done in any of a variety of ways, as would be readily understood by one of skill in the art. For example, if the titration reagent is detected by absorbance of a chromophore, the absorbance can be used to quantify the amount of titration reagent using Beer's Law, which states that there is a linear relationship between absorbance and concentration of chromophore. Thus, concentration of reagent can be determined using the absorbance coefficient of the chromophore, if that is known. Alternatively, a standard of known concentration can be prepared, and the absorbance of bound titration reagent determined by comparison with the standard. This is the method used in the Examples herein. For example, quantification of a UV chromophore by means of a standard can conveniently be accomplished using flow injection analysis on an HPLC connected to a UV detector. The standard and sample should be tested at a concentration wherein the relationship between absorbance and concentration is linear, which can be determined by means known to one of skill in the art. If necessary, the proper concentration can be achieved by dilution of standard and/or sample, where the calculation of the quantity of titration reagent will of course take any dilutions into account.

In a particularly preferred embodiment of the invention, the titration reagent comprises an alkyl ammonium group (e.g., benzylammonium chloride) and methanol, the silica surface is washed with methanol after the titration reagent has been allowed to react with substantially all the reactable silanol groups on the surface, the bound titration reagent is eluted from the capillary in a basic desorption solution (e.g., 0.1 M NaOH), and the amount of bound titration reagent eluted is determined by quantifying the absorbance of the eluted titration reagent, optionally by reference to a standard. The amount of reactable silanol groups is proportional to the amount of titration reagent eluted, e.g., is equal to the amount of titration reagent bound, or at least proportional to that amount.

Having now generally described the invention, the same will be more readily understood through reference to the following examples, which are provided by way of illustration, and are not intended to be limiting of the present invention, unless so specified.

### EXAMPLES

The following preparations and examples are given to enable those skilled in the art to more clearly understand and practice the present invention. They should not be construed as limiting the scope of the invention, but merely as being illustrative and representative thereof.

### Example 1

Calculation of Theoretical Concentration of Silanol Sites the Channel Surface of a Fused Silica Capillary

The concentration of silicon atoms on the channel surface of fused silica tubing can be calculated knowing the molecular formula of the fused silica (SiO₂), and the average bond distance between the silicon and oxygen atoms. Assuming that the surface is perfectly flat, the concentration of silicon atoms at the surface of fused silica glass is 8 μmoles/m². Assuming that each silicon group is in the silanol (SiOH) form, the maximum silanol concentration is 8 μmoles/m². In practice, the surface is not perfectly flat, and this can lead to observed concentrations that exceed the theoretical limit.

A capillary with 200 μm i.d. and 1 meter length has an inside surface area of 6.28 cm². Assuming a flat surface and 100% coverage of silanol groups, the highest theoretical capacity of a capillary with these dimensions is 5.0 nanomoles. However, it is unlikely that every silica atom is in the silanol form making the concentration of available reaction sites lower. Also, it is unlikely that the surface is perfectly flat, especially after a base etching treatment. An uneven or rough surface would increase the number of silanol sites on the surface.

### Example 2

Quantification of Silanol Groups on Etched and Non-Etched Capillary Surfaces

Two 1 meter sections of 200 um i.d., 360 um o.d. fused silica capillary tubing (supplied by Polymicro, Inc.) were etched by exposure of the inner surface of the capillary to 0.1 M NaOH for about 45 minutes. This was accomplished by flushing 0.1 mL of 0.1 M NaOH through the capillary at 0.1 mL/min, followed by 0.45 mL of 0.1 M NaOH at 0.01 mL/min. After etching, the capillary was flushed with about 1 mL DI water, followed by 0.1 mL dilute HCl, followed by about 2 mL DI water until the water coming out of the capillary was neutral pH. The same procedure was used to etch another two 1 meter sections of 200 um i.d., 360 um o.d. fused silica capillary tubing obtained from a different supplier (SGE, Inc., Ringwood, Australia).

The following day, the four sections of etched capillary (two each from SGE and Polymicro) were treated with benzyltrimethylammonium (BTA), which involved flushing each capillary with 0.5 mL of a 100 mM solution of BTA in methanol, at a flow rate of 0.1 mL/min. The methanol solvent ensures that the interaction of BTA with the fused silica wall is only electrostatic and not adsorptive. The capillaries were then flushed with 1 mL methanol at a rate of 0.25 mL/min, after which any methanol remaining in the capillary was blown out with nitrogen gas at 30 psi. A 10 μL slug of 0.1 M NaOH (desorption solution) was then taken up into the capillary to desorb and collect the BTA. The slug of desorption solution is passed back and forth through the entire length of the capillary 3 times prior to expulsion from the capillary and collection. 0.1 M NaOH extracts the BTA quickly; however, 0.1 M HCl and PBS solution can alternatively be used.

The 10 μL slug of desorbed BTA was quantified by flow injection analysis with a water carrier and UV detection at 254 and 266 nm using an HP 1050 HPLC (Hewlett-Packard, Palo Alto, Calif.). The samples are compared to standards prepared by diluting 0.5 mL of the BTA solution to 100 mL in methanol (0.5 mM, or 5 nmole per 10 μL).

Two sections of 200 um i.d., 360 um o.d.×1 m fused silica capillary tubing (supplied by Polymicro, Inc.) and two sections of 200 um i.d., 360 um o.d.×1 m fused silica capillary tubing (supplied by SGE, Inc.) that had not been etched were then treated with BTA and assayed for silanol groups in the same manner. Thus, in total eight sections of capillary were tested—two non-etched from SGE (SN1 and SN2), two etched from SGE (SE1 and SE2), two non-etched from Polymicro (PN1 and PN2), and two etched from Polymicro (PE1 and PE2).

The entire experiment described above was then repeated two more times; each time eight sections of capillary were tested, for a total of 16 sections—SN3, SN4, SN5, and SN6 (non-etched capillary from SGE); SE3, SE4, SE5, and SE6 (etched capillary from SGE); PN3, PN4, PN5, and PN6 (non-etched capillary from Polymicro); and PE3, PE4, PE5, and PE6 (etched capillary from Polymicro).

In the three experiments, a total 24 capillaries were analyzed for silanol group quantity. The results are presented below in Table 1. Thus, for each of the 4 types of capillaries (SN, SE, PN and PE), there are six corresponding data entries, e.g., SN1-SN6. The data is the amount of BTA desorbed from the column (in nmols), which is a measure of silanol concentration.

TABLE 1*

| Capillary ID | 1 | 2 | 3 | 4 | 5 | 6 |
|---|---|---|---|---|---|---|
| SN | 63.3 | 71.4 | 63.7 | 90.6 | 28.6 | 69.8 |
| SE | 4960 | 6050 | 4570 | 4390 | 3540 | 2750 |
| PN | 6.5 | 16 | 56.2 | 4.7 | 6.5 | 8.8 |
| PE | 17.8 | 4.0 | 12 | 10 | 9.7 | 15.2 |

*BTA recovered from capillary in nmols

Assuming 100% coverage of silanol groups and a flat surface, the maximum concentration of BTA is 5 nmoles/10 μL of solvent. The data shows that there are substantial differences in the number of silanol groups. In particular, it is apparent that for the non-etched capillaries, there are considerably more silanol groups than would be predicted for a theoretical flat surface. Furthermore, there are substantial differences in silanol concentration in capillaries obtained from different vendors. Interestingly, the results show that etching of the SGE capillaries results in a dramatic increase in the number of reactable silanol groups, while etching has much less effect on the Polymicro capillaries.

While the invention has been described in connection with specific embodiments thereof, it will be understood that it is capable of further modifications and this application is intended to cover and variations, uses, or adaptations of the invention that follow, in general, the principles of the invention, including such departures from the present disclosure as come within known or customary practice within the art to which the invention pertains and as may be applied to the essential features hereinbefore set forth. Moreover, the fact that certain aspects of the invention are pointed out as preferred embodiments is not intended to in any way limit the invention to such preferred embodiments.

1. A method for measuring the concentration of reactable silanol groups on a silica surface comprising the steps of:
f) contacting a silica surface with a silanol titration solution, wherein said titration solution comprises a detectable and quantifiable titration reagent that binds with substantially all reactable silanol groups on the silica surface;
g) allowing said titration reagent to bind substantially all of the silanol groups on the silica surface;
h) removing the silanol titration solution from the silica surface, along with substantially all of the titration reagent that has not bound to a reactable silanol group, under conditions where titration reagent that has bound to a silanol group remains bound;
i) detecting and quantifying the bound detection reagent, after optionally eluting the bound detection reagent from the silica surface; and
j) determining the concentration of reactable silanol groups on said silica surface from the quantity of bound titration reagent.

2) The method of claim 1, wherein the titration reagent binds substantially all reactable silanol groups on the silica surface in a manner that is substantially stoichiometric, and wherein the known binding stoichiometry between said titration reagent and reactable silanol groups is used to calculate the concentration of reactable silanol groups on said silica surface.

3) The method of claim 2, wherein the binding stoichiometry between the titration reagent and the reactable silano groups is known.

4) The method of claim 3, wherein the binding stoichiometry is 1:1.

5) The method of claim 1, wherein the silica surface comprises fused silica.

6) The method of claim 1, wherein the silica surface comprises the channel surface of a capillary.

7) The method of claim 1, wherein said titration reagent binds to said reactable silanol groups through an ionic interaction.

8) The method of claim 1, wherein said titration reagent is small enough such that the binding of a first titration reagent to a reactable silanol group on said silica surface does not substantially block any other reactable silanol groups on said silica surface from reaction with a second titration reagent.

9) The method of claim 1, wherein said titration reagent has a MW of less than 500 Da.

10) The method of claim 1, wherein said titration reagent comprises a quaternary alkyl ammonium group.

11) The method of claim 10, wherein said titration reagent is benzyltrimethylammonium.

12) The method of claim 1, wherein said titration reagent comprises a chromophore which is used to detect and quantify the bound titration reagent, optionally after elution of the reagent from the silica surface.

13) The method of claim 12, wherein said chromophore absorbs UV radiation.

14) The method of claim 1, wherein the binding reaction between said titration reagent and a reactable silanol group is reversible, and wherein bound titration reagent is eluted from the silica surface between steps (c) and (d).

15) The method of claim 1, wherein the silica surface is washed to substantially remove the silanol titration solution from the silica surface prior to detection and quantification of bound titration reagent.

16) The method of claim 14, wherein said titration reagent comprises a quaternary alkyl ammonium group and a UV chromophore, wherein the silica surface is washed to substantially remove the silanol titration solution from the silica surface prior to detection and quantification of bound titration reagent, and wherein the titration reagent is detected by absorbance of UV light.

17) The method of claim 16, wherein said titration reagent is benzyltrimethylammonium.
